(19) United States
(12) Patent Application Publication
(10) Pub. No.: US 2017/0303805 A1
(43) Pub. Date: Oct. 26, 2017

(54) Method and Apparatus for Simulating the Wrist Pulse Patterns for Pulse Diagnosis

(71) Applicant: Mona Boudreaux, Wonder Lake, IL (US)

(72) Inventor: Mona Boudreaux, Wonder Lake, IL (US)

(73) Assignees: Mona Boudreaux, Wonder Lake, IL (US); Ying Sun, West Warwick, RI (US); Gloria Faye Boudreaux-Bartels, West Kingston, RI (US)

(21) Appl. No.: 15/134,683

(22) Filed: Apr. 21, 2016

(51) Int. Cl. A61B 5/024; A61B 5/00

(52) U.S. Cl. CPC A61B 5/02438; A61B 5/6824; A61B 5/7225; A61B 5/0015; A61B 5/4854; A61B 2562/02

(57) ABSTRACT

A method and an apparatus are disclosed for simulating the wrist pulse patterns to be used for teaching and practicing pulse diagnostic techniques in traditional Chinese medicine (TCM) and other alternative medicines. The method represents the wrist pulse patterns and artery responses by use of six characteristic qualities: width, depth, strength, rhythm, length, and propagation. One embodiment of the invention uses a processor to drive three solenoids. The three plungers of the solenoids produce time-varying forces that simulate the wrist pulse waves felt by the user's fingers when evaluating pulse patterns in humans or animals. Via a force sensor, the processor detects the compression force from the palpating fingers and classifies said force into one of the three ranges (shallow, middle, and deep). A purity of pulse waveforms representing the various pulse patterns defined in TCM are pre-programmed into the processor in terms of their characteristic qualities and compression forces. The width of the artery is represented by either a width-adjustable plunger head or a multi-lumen tube placed on top of the plungers. Once the user selects a specific pulse pattern, the device continuously generates the pulse waveforms that change dynamically in response to the compression force.

### CROSS-REFERENCE TO RELATED APPLICATION

Under 37 CFR 1.53 §1.81(a3), this U.S. full utility patent application is hereby converted from the U.S. provisional patent application filed on Apr. 23, 2015 (application no. 62178949) entitled “Method and apparatus for simulating the wrist pulse patterns for pulse diagnosis” by inventors Mona Boudreaux, Ying Sun, and G. Faye Boudreaux-Bartels.

### REFERENCES

### U.S. Patent Documents

| | |
|---|---|
| 5,027,641 | L. F. Costello, Jr. Oscillometric non-invasive blood |
| | pressure simulator. 1991. |
| 5,170,796 | I. Kobayashi. Pulse wave detecting apparatus. 1992. |
| 5,381,797 | S. C. Pak, et al. Pulse diagnostic device and method |
| | of measuring a pulse wave using this device. 1995. |
| 6,767,329 B2 | K. Amano, et al. Diagnostic apparatus for analyzing |
| | arterial pulse waves. 2004. |
| 7,510,398 B1 | W. E. Thornton. Apparatus for simulating a pulse and |
| | heart beat and methods for using same to train |
| | medical professionals. 2009. |
| 7,972,141 B2 | G. J. Morris. Blood pressure simulation apparatus with |
| | tactile feedback. |
| 8,317,716 B2 | J. Y. Kim, et al. Diagnosis system of deficient and |
| | forceful pulse. 2012. |

### Foreign Patent Documents

| | |
|---|---|
| WO 2003073932 A1 | H. H. Huang. Pulse diagnostic system. 2003. |
| WO 2009019720 A2 | A. Bhat, et al. A non-invasive device nadi |
| | tarangini useful for quantitative detection |
| | of arterial nadi pulse waveform. 2009. |
| EP 2 523 135 A1 | A. J. Elkefjord. Medical simulation system. 2012. |

### Other Publications

S. Walsh and E. King. Pulse Diagnosis: A Clinical Guide. 1st edition. Publisher: Churchill Livingstone. December 2007.
B. Flaws. The Secret of Chinese Pulse Diagnosis. 2nd edition. Boulder, Colo.: Blue Poppy Press. March 1997.
S. McLellan, C. Liese, M. Andrews, M. Boudreaux, G. F. Boudreaux-Bartels, E. Chabot, and Y. Sun. A Microprocessor-based wrist pulse simulator for pulse diagnosis in traditional Chinese medicine. 40th Northeast Bioengineering Conference, Boston, Mass., April 25-27, 2014.
J. Maestri, S. Borges, G. Halkidis, M. Boudreaux, G. F. Boudreaux-Bartels, and Y. Sun. Graphical user interface to generate waveforms for a wrist pulse simulator used in traditional Chinese medicine education. 42nd Northeast Bioengineering Conference, Vestal, N.Y., Apr. 5-7, 2016.

### BACKGROUND OF THE INVENTION

Pulse diagnosis is an important technique for diagnosing the health conditions and the course of treatment based on pulse patterns detected at the wrist in traditional Chinese medicine (TCM) as well as other alternative medicines, such as ayurvedic medicine, traditional Mongolian medicine, Siddha medicine, traditional Tibetan medicine, and Unani. The pulses are felt by pressing the index finger, the middle finger, and the ring finger against the radial artery at the wrist of the subject. Pulse diagnosis is noninvasive, convenient, inexpensive, quick, and painless. Pulse diagnostic techniques are taught at schools that teach TCM, Ayurveda, and acupuncture. The training of pulse diagnostic techniques is highly hands-on and tactile. It is often difficult to find patients who exhibit a full range of symptoms for students to practice on and learn from. Thus, the purpose of the present invention is to provide a method and a simulation device for training people how to perform pulse diagnosis.

In the USA there are over 45,000 acupuncturists who use pulse diagnosis to find where there are problems with the flow of energy in the body. Each year 3,500 students of TCM undergo licensing exams administrated by organizations such as the National Certification Commission for Acupuncture and Oriental Medicine (NCCAOM). Naturopaths, napropaths, medical doctors, chiropractors, and veterinarians also perform acupuncture and pulse diagnosis. There are many more practitioners of TCM in other parts of the world, especially in Asia. The present invention leads to a product to be used by schools, teachers, and students for teaching, learning, and testing the techniques of pulse diagnosis.

### DESCRIPTION OF THE RELATED ART

In Western medicine, the study of the pulse is known as sphygmology. The pulse is palpated over an artery near the surface of the body by pressing a finger or two fingers against a bone. The pulse can be detected from the carotid artery at the neck, the brachial artery on the inside of the elbow, the radial artery at the wrist, the femoral artery at the groin, the popliteral artery behind the knee, the posterior tibial artery near the ankle joint, and dorsalis pedis artery on the foot. A typical clinical pulse palpation is conducted by pressing the index and middle fingers on the radial artery at the wrist or the carotid artery at the neck. A stop watch is used to count the number of pulses for a certain duration, say 15 seconds, and the heart rate in beats per minute (bpm) is given by the count multiplied by 4. The sole purpose of pulse palpation in Western medicine is to measure the heart rate; it is not intended to extract any other health-related information.

By contrast, in traditional Chinese medicine (TCM) and other alternative medicines, pulse palpation is used to extract much more diagnostic information about the patient's health conditions. The pulse palpation in TCM differs from that in Western medicine in the following respects:
1. In TCM, the pulse palpation is always performed at the wrist on humans, and for both the right hand and the left hand.
2. In TCM, three fingers instead of two are used to palpate at three different positions. The first position closest to the wrist is the can (inch), the second guan (gate), and the third pulse position furthest away from the wrist is the chi (foot).
3. Furthermore, the pulse is palpated at shallow, middle, and deep levels by changing the compression force with the fingers.
4. The conditions of the internal organs are mapped to and reflected at the different positions. On the left hand, the first position represents the heart and small intestine; the second position, the liver and gallbladder; and third position, the kidney yin and bladder. On the right hand, the first position represents the lungs and large intestine; the second position, the spleen and stomach; and the third position, the kidney yang and uterus or triple burner.
5. In addition to the rhythm, many other pieces of information related to the strength and other qualities of the pulse are detected.

William. E. Thornton (U.S. Pat. No. 7,510,398 B1) disclosed an apparatus that uses an electronic tactile pulse simulator to generate pressure pulses simulating arterial pressure pulses discernible by touch. Thornton's invention is useful for teaching pulse palpation in Western medicine. However, it lacks the multiple tactile outputs and the sophistication of representing different pulse patterns for teaching the pulse diagnostic techniques in TCM and other alternative medicines.

Simulating the wrist pulse patterns for TCM is the inverse problem of detecting the wrist pulse patterns. There exist an abundance of prior art for detecting wrist pulses, for example: U.S. Pat. No. 5,170,796, U.S. Pat. No. 5,381,797, U.S. Pat. No. 6,767,329 B2, U.S. Pat. No. 8,317,716 B2, WO 2003073932 A1, and WO 2009019720 A2. However, there is no prior art of simulating the wrist pulse patterns for TCM that can be found. The classification of the pulse patterns in TCM has not yet been standardized. Generally speaking, experts of pulse diagnosis have described 28 different patterns relating to internal energy flows and various diseases conditions. The present invention is concerned with 1) an effective method for representing the characteristic qualities of the wrist pulse patterns, and 2) a realization of pulse simulator for teaching pulse diagnostic techniques.

### BRIEF SUMMARY OF THE INVENTION

This invention discloses a novel method and a device for simulating the palpation of wrist pulses. All 28 wrist pulse patterns in the traditional Chinese medicine can be represented by use of 6 characteristic qualities: width, depth, strength, rhythm, length, and propagation. These 6 characteristic qualities guide the design of a set of wrist pulse waveforms, which are pre-programmed in a processor to deliver time-varying tactile outputs against the palpating fingers via plungers of three solenoids. The wrist pulse waveforms reacts dynamically to the compression force from the palpating fingers measured with a force sensor. The palpation sense of the artery width is controlled by either width-adjustable plungers or a multi-lumen flexible tube. The field of use of the wrist pulse simulator device includes 1) teaching pulse diagnostic techniques in traditional Chinese medicine and other alternative medicines, and 2) developing and testing automated pulse diagnostic devices.

### BRIEF DESCRIPTION OF THE TABLES

TABLE 1 summarizes the 28 wrist pulse patterns, the interpretations and health relevancies in TCM for the categories of (A) floating and sinking pulses, (B) slow and rapid pulses, and (C) feeble and replete pulses.

TABLE 2 shows the six characteristic qualities of the wrist pulses and how the simulator represents them.

### BRIEF DESCRIPTION OF THE DRAWINGS

The following description of the figures may be further understood with reference to the accompanying drawing in which:

FIG. 1 is an illustrative diagrammatic view of an embodiment of the wrist pulse simulator device that produces tactile outputs via three solenoids under the control of a processor.

FIG. 2 is a block diagram of an embodiment of the wrist pulse simulator device showing the major components and the signal paths.

FIG. 3 shows an embodiment for representing the width of the artery by using a magnetically actuated rod to lock the plunger in the wide configuration (A) or in the narrow configuration (B).

FIG. 4 shows a different embodiment for representing the width of the artery by using a three-lumen flexible tube. A wide artery is represented by inflating all three lumens; a narrow artery is represented by inflating only the central lumen.

FIG. 5 shows the processes of specifying the pulse waveforms for the various wrist pulse patterns either by using a graphical waveform designer or by acquiring realistic waveforms recorded from human subjects and recording numerical representations of the waveforms.

FIG. 6 shows experimental data of changing the pulse waveform magnitude in response to the compression force.

FIG. 7 shows experimental data of three hand-drawn wrist pulse patterns and the corresponding waveforms outputted by the digital-to-analog converters for driving the solenoids.

### DETAILED DESCRIPTION OF PREFERRED EMBODIMENT

Table 1 summarizes the 28 pulse patterns in TCM, which are classified into 6 categories: floating and sinking pulses (Table 1A), slow and rapid pulses (Table 1B), and feeble and replete pulses (Table 1C). For each pulse pattern, its TCM interpretation and health relevancy are also given in Table 1. The present invention discloses the use of 6 characteristic qualities to represents the wrist pulse patterns and artery responses. These characteristic qualities are as follows.

a) width: thin or wide artery,

b) depth: superficial or deep artery,

c) strength: forceful or forceless pulse,

d) rhythm: fast or slow rate; rhythmic or arrhythmic pulses,

e) length: short or long duration of the contraction, and

f) propagation: delay and magnitude change among the three positions.

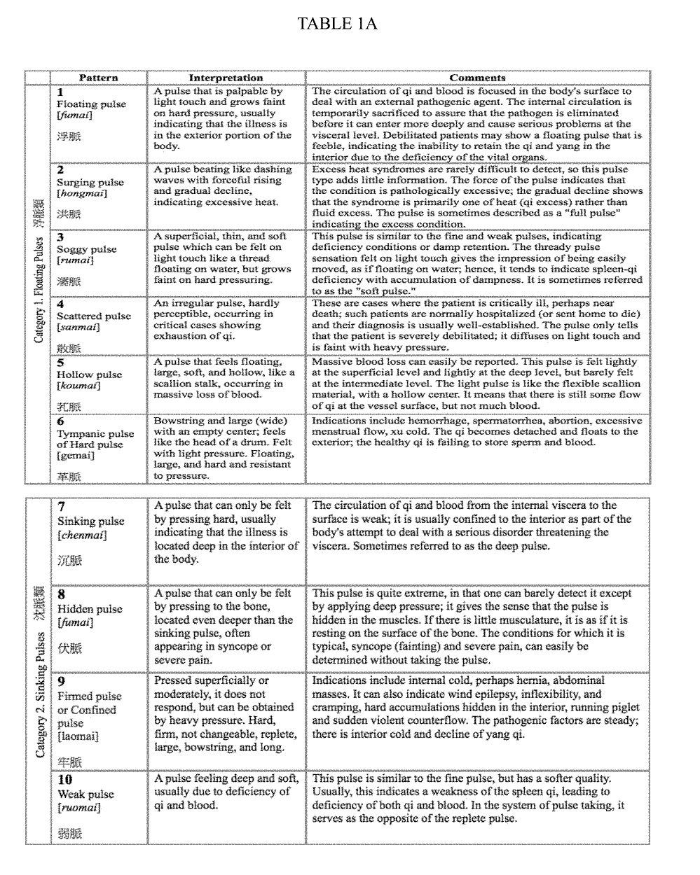

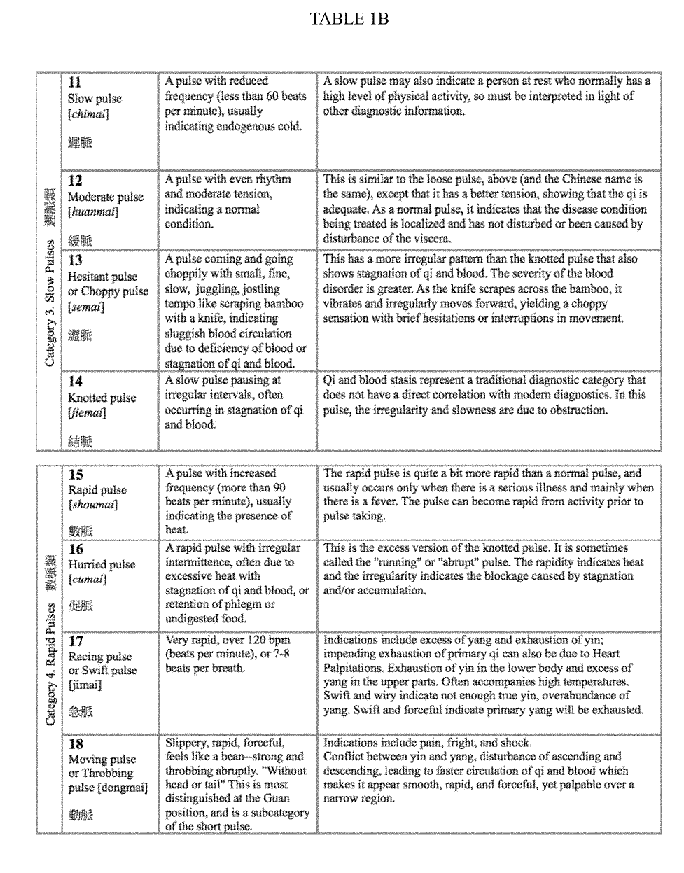

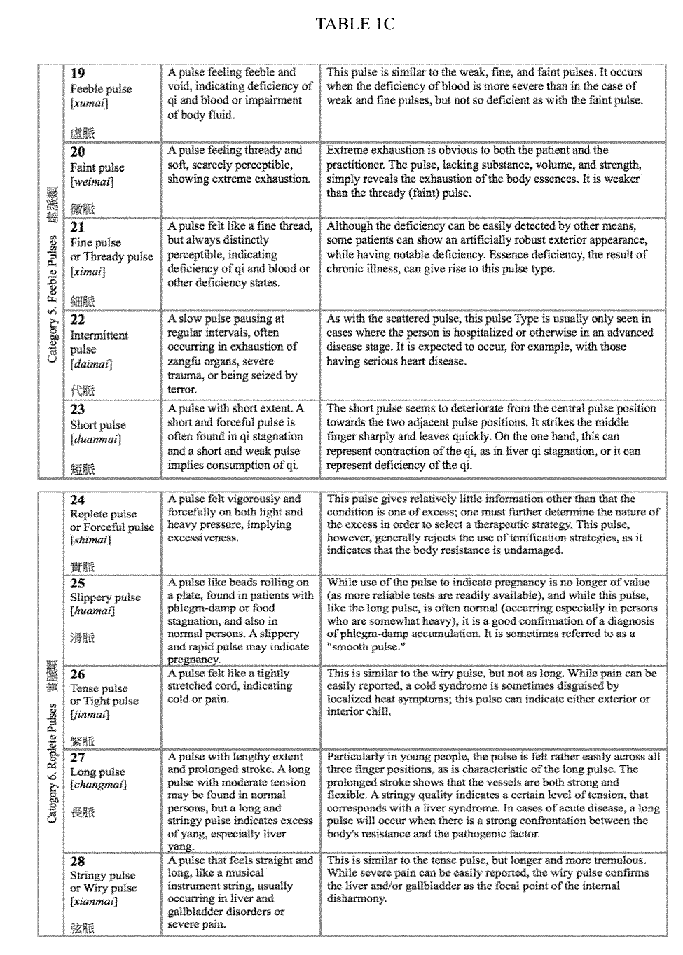

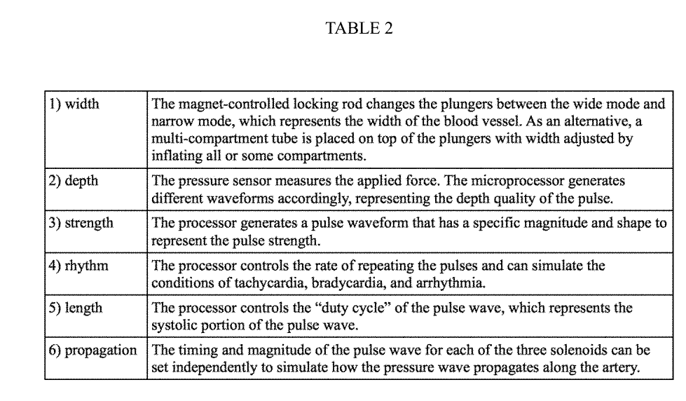

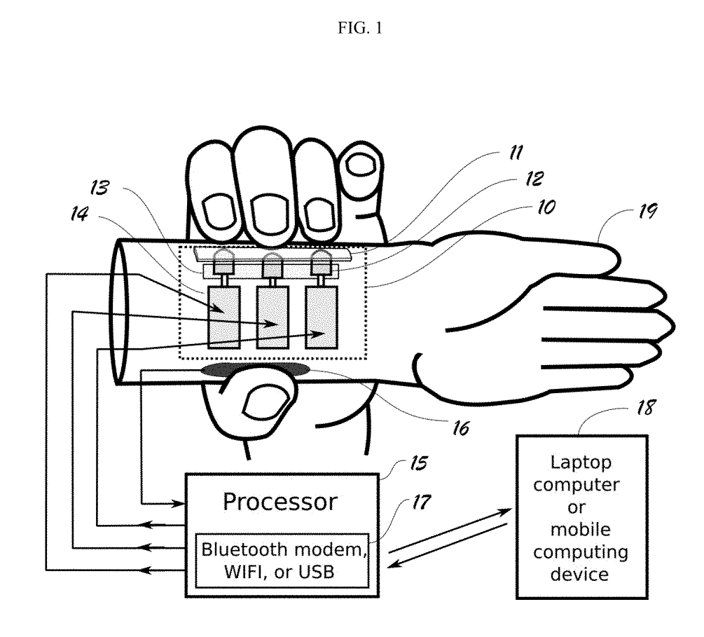

FIG. 1 is an illustrative diagrammatic view of an embodiment of the pulse simulation. The pulse simulator 10 is a hand-held device that transfers tactile outputs to the fingers via a soft pad 11 made of silicone rubber or a similar material. The three moveable plungers 12 underneath the soft pad are used to simulate the wrist pulses. A width control unit 13 is used to change the width of the plungers, which will be described in more detail later. The plungers are actuated by three solenoids 14, which are controlled by a processor 15. The hand-held unit has a force sensor 16 to detect the force applied by the thumb, which indirectly measures the compression force applied by the three palpating fingers against the soft pad.

The processor system 15 contains a communication unit 17, which can receive data from or transmit data to a laptop computer or a mobile computing device 18 such as a smartphone or a tablet. The communication unit 17 can be either wireless (Bluetooth or WIFI modem) or wired (USB).

As an option, the hand-held pulse simulator 10 can be embedded in a life-sized hand-wrist model 19. The model is anatomically correct in terms of its dimensions and the artery position to provide a more realistic simulation. The three moveable plungers 12 are positioned near the surface and underneath a thin soft pad 11 around the wrist area to deliver the pulses. The user's hand is applied to the pulse simulator with the index, middle and ring fingers pressing onto the three plungers 12, respectively. The thumb is at the opposite side of the wrist where the force sensor 16 is positioned.

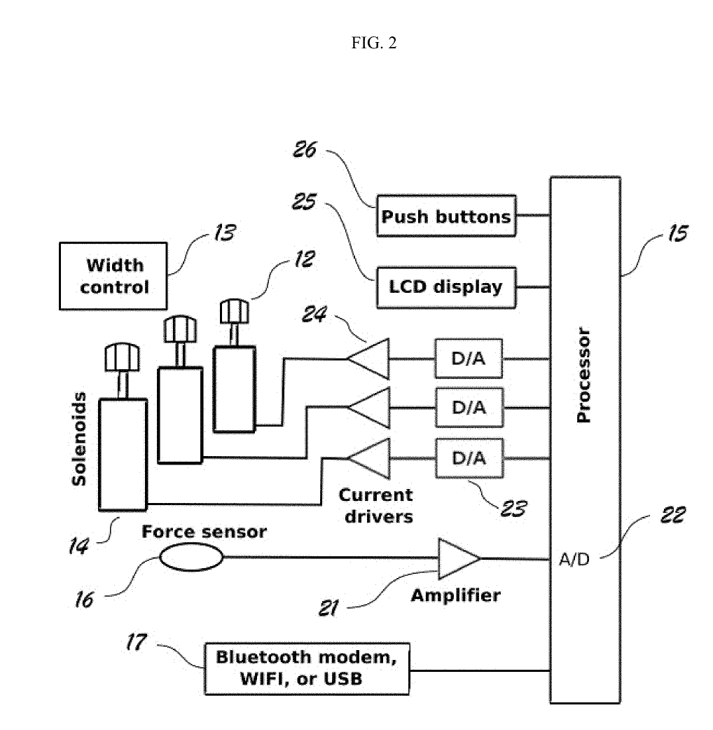

FIG. 2 further specifies the functional units and signal paths of the pulse simulator in FIG. 1. The compression force from the user's hand is sensed by a force sensor 16, which is conditioned by an amplifier 21 and acquired by a processor 15 via an analog-to-digital converter 22. The compression force is compared to two pre-set thresholds to determine which level (shallow, middle, or deep) the pulse is palpated at. The digital pulse waveforms and parameters pertaining to the characteristic qualities are stored in the processor 15. The user can select which of the pre-programmed pulse patterns to simulate via a user interface facilitated by an LCD display 25 and push buttons 26. The pulse waveforms are played back in response to the compression force. The stored digitized waveforms are sent to three analog-to-digital converters 23 that actuate three solenoids 14 via current drivers 24. The three plungers 12 of the solenoids deliver the tactile outputs to be palpated by the user's fingers. A width control unit 13 provides a means of adjusting the sensation of either a wide artery or a narrow artery.

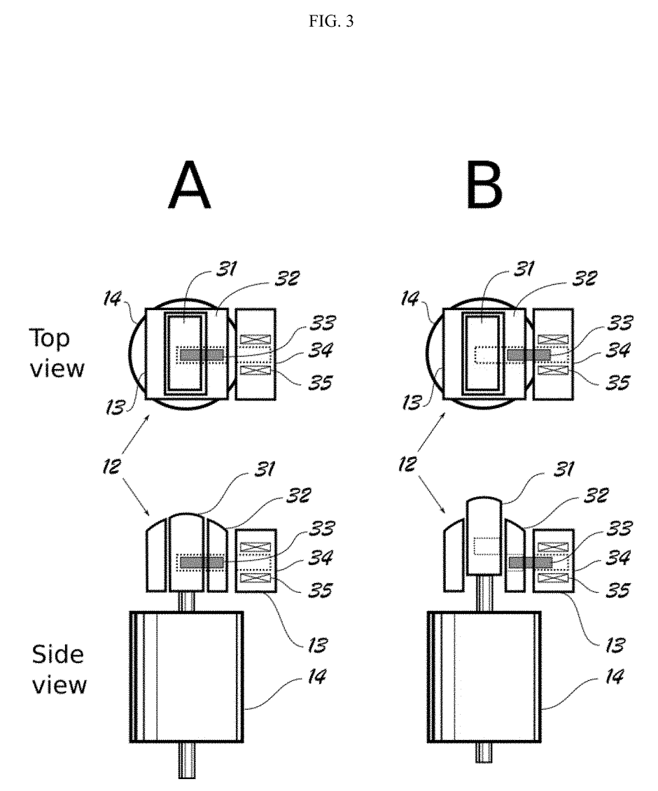

FIG. 3 shows a possible embodiment for adjusting the width sensation. In FIG. 3 top and side views, a plunger consists of a center piece 31 surrounded by a side piece 32. The plunger assembly is connected to the solenoid 14, which moves the plunger up and down in proportion to the supplied electrical current. A locking rod 33 can slide through the center piece 31, the side piece 32, and a separate actuator unit 33. The locking rod 33 is made of a ferromagnetic material such that it can be pushed or pulled by changing the polarity of an electromagnet 35 embedded in the actuator unit 34. FIG. 3A (left column) shows the plunger in the wide configuration, in which the locking rod 33 is pushed to a position between the center piece 31 and the side piece 32. With this wide configuration the center piece 31 and the side pieces 32 move up and down together as one unit to simulate the effect of a wide blood vessel. FIG. 3B (right column) shows the plunger in the narrow configuration, in which the locking rod 33 is pulled back to a position between the side piece 32 and the actuator unit 34. With this narrow configuration, only the center piece 31 moves up and down to simulate the effect of a narrow blood vessel.

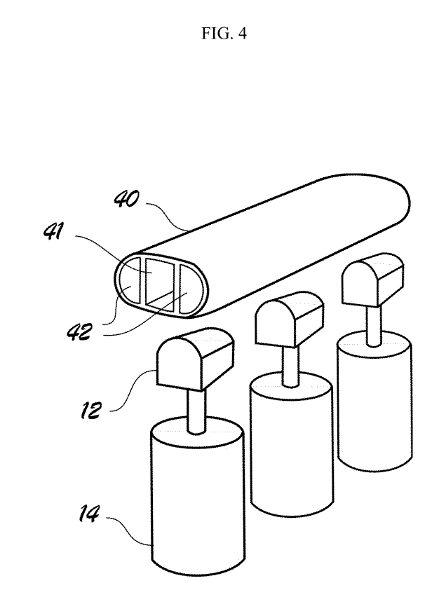

FIG. 4 shows an alternative embodiment for representing the width of the artery by employing a three-lumen tube 40. The tube has a center lumen 41 and two side lumens 42. A wide artery is represented by inflating all three lumens. A thin artery is represented with the center lumen inflated and the two side lumens deflated. The inflation mechanism (not shown) can be either pneumatic (air) or hydraulic (water). The forces from the plungers are transferred to the fingers through the three-lumen tube 40. Thus, the user can palpate the difference of the simulated width of the artery.

Table 2 summarizes how the pulse simulator uses the aforementioned hardware and software methods to represent the 6 characteristic quantities of the wrist pulse patterns. For each of the 28 pulse patterns given in Table 1, the simulator stores the following information: 1) a digital waveform or waveforms representing the time course of the pulse, 2) a set of 6 characteristic quantities pertaining to this pulse pattern, and 3) how the pulses react to the shallow, middle, and deep levels of compression.

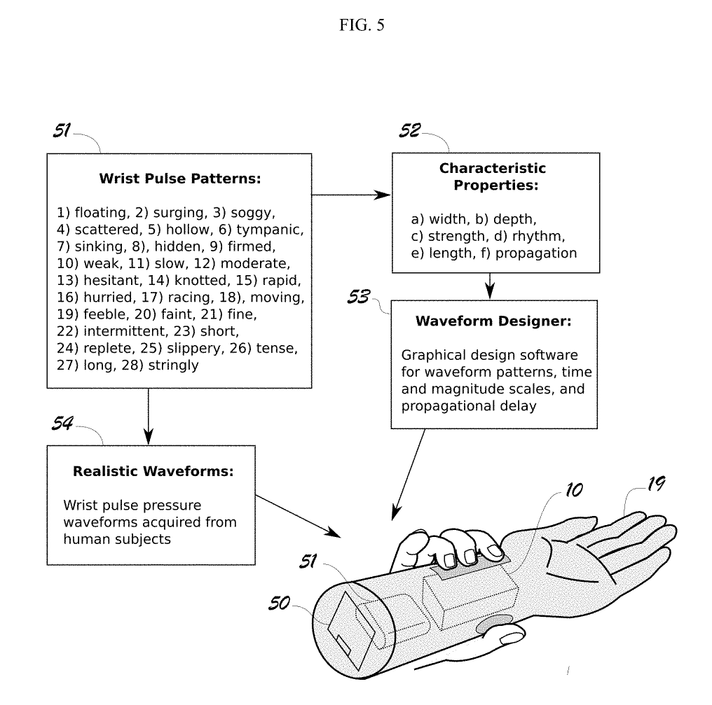

FIG. 5 shows the processes of specifying/designing the pulse waveforms to be downloaded to the wrist pulse simulator 10. The wrist pulse simulator 10 can be incorporated into the wrist-hand model 19 as a single integrated unit. An access trap cover 50 is available for installing/replacing the batteries 51. The waveform design process begins with the selection of one of the wrist pulse patterns 51. There are at least two possible ways to design the pulse waveforms. One way is to first extract the 6 characteristic properties 52 for the chosen wrist pulse pattern. Then, a waveform designer 53 is used to design a set of waveforms for the three solenoids and the three levels of the compression force. The waveform designer 53 is a software system that runs on a laptop computer or a mobile computing device. The pulse waveforms can be drawn by hand with a graphical user interface or generated by equations. Additional parameters such as the time delays among the three solenoids and the magnitude/waveform changes in response to the finger compression forces can be specified. This set of waveforms can then be downloaded to the wrist pulse simulator 10 for execution.

Another way to specify the wrist pulse waveforms is to use an array of pulse pressure sensors and a data acquisition system 54 to record realistic waveforms from human subjects. These waveforms are scaled to the proper magnitude ranges and downloaded to the pulse simulator 10 for execution.

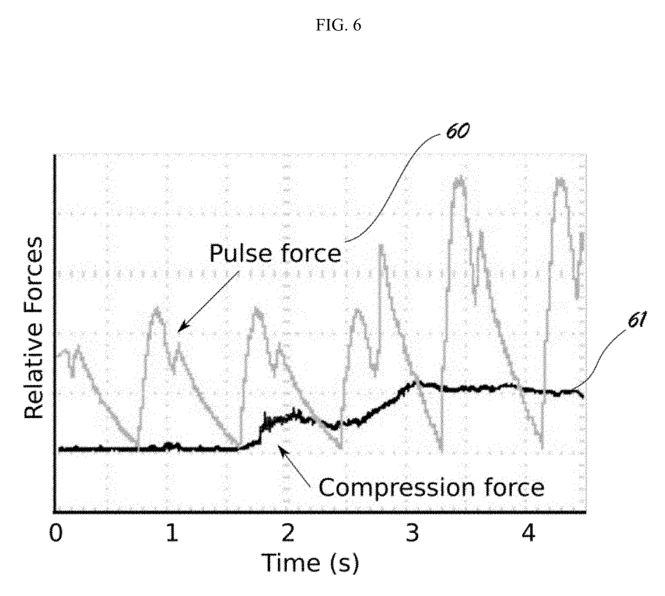

A functional prototype of the pulse simulator has been built to verify that the design concept and specifications are realizable. FIG. 6 shows the experimental data of changing the magnitude of the pulse force waveform 60 in response to the compression force 61. This is to simulate the situation of applying different levels of forces (shallow, middle, and deep) through the palpating fingers. The artery reacts to the given compression force and produces the pulse waveform accordingly.

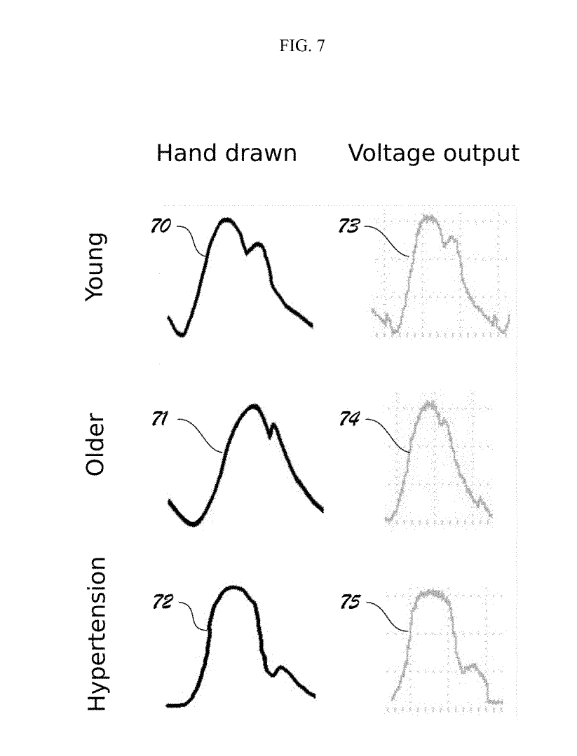

FIG. 7 shows the experimental data of three hand-drawn wrist pulse patterns by use of the waveform designer representing a young person 70, an older person 71, and a person with hypertension 72. These waveforms were downloaded to the pulse simulator for execution. The corresponding waveforms 73-75 outputted by the digital-to-analog converters for driving the solenoids showed a good resemblance of the original hand-drawn waveforms.

There are 3 independent claims and 7 dependent claims in this invention. The claims structure is as follows:
1. Method for wrist pulse simulation (independent)
   2. Pulse patterns represented with 6 characteristic qualities
   3. Processor to play back pulse waveforms through solenoids
   4. Mechanical means of changing the palpation of the artery width
   5. Pulse patterns in response to compression force
6. Waveform designer and data acquisition for generating wrist pulse patterns (independent)
7. Apparatus for wrist pulse simulation (independent)
   8. Mechanical means of changing the palpation of the artery width
   9. Adjustment of wrist pulse waveforms in response to the compression force
   10. Pulse pattern generation system linked to the wrist pulse simulator

What is claimed is:

1. A method for simulating the palpation of wrist pulses that comprises the steps of
a) representing the wrist pulse patterns and artery responses with a set of characteristic qualities;
b) implementing the characteristic qualities with a set for force waveforms delivered by a plurality of solenoids under the control of a processor;
c) changing the palpation of the artery width by mechanical means; and
d) delivering the wrist pulse patterns in response to the compression force of the palpating fingers.

2. In the method for simulating the palpation of wrist pulses according to claim 1, said characteristic qualities of the wrist pulse patterns and artery responses include width, depth, strength, rhythm, length, and propagation.

3. In the method for simulating the palpation of wrist pulses according to claim 1, said pulse waveforms are pre-stored in the said processor and selectively played back in real-time via a plurality of digital-to-analog converters to drive said solenoids.

4. In the method for simulating the palpation of wrist pulses according to claim 1, said mechanical means of changing the palpation of the artery width employs either a plurality of plungers with adjustable width attached to said solenoids or a multi-lumen flexible tube placed between said solenoids and the palpating fingers.

5. In the method for simulating the palpation of wrist pulses according to claim 1, said compression force of the palpating fingers is sensed by said processor via a force sensor and controls the delivery of said wrist pulse patterns.

6. A method of generating the wrist pulse waveforms by use of either
a) a waveform designer software system to draw the waveforms by hand via a graphical user interface or to generate the waveforms with equations; or
b) a data acquisition system to measure the realistic wrist pulse patterns from human subjects via an array of pulse pressure sensors placed around the wrist area.

7. An apparatus for simulating the palpation of wrist pulses that comprises the components of
a) a processor to store and play back wrist pulse waveforms;
b) a plurality of solenoids to deliver the pulse waveforms to the palpating fingers;
c) a mechanical means of changing the palpation of the artery width;
d) a force sensor to measure the compression forces of the palpating fingers; and
e) a live-sized wrist-hand model to enclose the aforementioned components; and
f) a wrist pulse pattern generation system to design the pulse waveforms by drawing, computing from equations, and/or acquiring realistic waveforms from human subjects.

8. In the apparatus for simulating the palpation of wrist pulses according to claim 7, said mechanical means of changing the palpation of the artery width employs either a plurality of plungers with adjustable width attached to said solenoids or a multi-lumen flexible tube placed between said solenoids and the palpating fingers.

9. In the apparatus for simulating the palpation of wrist pulses according to claim 7, an algorithm is implemented in said processor to adjust the outputs of the wrist pulse waveforms in response to the input of the compression force from said force sensor.

10. In the apparatus for simulating the palpation of wrist pulses according to claim 7, said wrist pulse pattern generation system is implemented on a laptop computer or mobile computing device connected to the processor in the wrist pulse simulator via a wireless or wired communication link.
